(19) United States
(12) Patent Application Publication
(10) Pub. No.: US 2006/0235274 A1
(43) Pub. Date: Oct. 19, 2006

(54) RIGID ENDOSCOPE OPTICS WITH A COMPOUND HOUSING

(75) Inventors: Thomas Forster, Molln (DE); Mathias Kraas, Haseldorf (DE); Gilbert Spahn, Schwarzenbeck (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/420,126

(22) Filed: May 24, 2006

Related U.S. Application Data
(63) Continuation-in-part of application No. 11/054,813, filed on Feb. 10, 2005.

(30) Foreign Application Priority Data
Feb. 26, 2004 (DE) 10200409383.0
Jul. 28, 2005 (DE) 102005035910.8

(51) Int. Cl. A61B 1/00

(52) U.S. Cl. 600/133

(57) ABSTRACT

A rigid endoscope optics (1) including a housing consisting of several housing parts (5, 8, 9, 6, 16) which hermetically encloses optic elements (3, 2, 4) and which is fitted with a hermetic connection (13, 14; 7, 15) between two housing parts (9,8; 9, 16) is characterized in that one (9) of the connected housing parts is made of PPS (polyphenylenesulfide) plastic or of PPSU (polyphenylsulfone) plastic, and the other (8, 16) connected housing part is made of PPS, PPSU or glass, the housing parts (8, 9, 16) being fitted with a metal coating (7, 13, 14, 15) at the connection site (13, 14; 7, 15) and are soldered to one another.

This application is a continuation-in-part of U.S. patent application Ser. No. 11/054,813, filed on Feb. 10, 2005 and currently pending, the entirety of which is incorporated herein.

### BACKGROUND OF THE INVENTION

Endoscope optics having several housing parts are protected by a hermetic housing against entry of water and in particular, also against entry of water vapor, and accordingly can be steam-sterilized before being used in surgery without the steam fogging optical surfaces.

In part, the housing consists of an elongated tube receiving an image guide and is sealed at the distal end by a hermetically sealed window. Housing parts are connected to the proximal tube end and they receive, within an inner space, further devices such as ocular lenses, pertinent, hermetically sealed windows, and the like.

The connections between the housing parts must be hermetic. Therefore, as regards known endoscopes of this type, the housing parts are metallic and welded or soldered to each other. The windows, which are parts of the enclosing housing, also are soldered into place. Effective hermetic junctions can only be made in this manner, namely by welding or soldering, whereas such junctions can be implemented adhesively only with great difficulties if at all.

This restriction to metals for the housing parts represents a drawback.

The German patent document 101 03 433 A1 describes a flexible endoscope wherein the ceramic parts and the glass parts of the surface metal coating are hermetically soldered together.

The German patent document 29 04 893 A1 shows a glass fiber being connected to a sleeve. The sleeve may be plastic or metallic, and, in the latter case, may be soldered to the metal-coated glass fiber.

The German patent document 37 19 382 A1 describes cold-compression welding of solder-coated optical components for the purpose of circumventing soldering.

The Japanese patent document 04-073691 A mentions using PEEK (polyether-etherketone) plastic.

### BRIEF SUMMARY OF THE INVENTION

The objective of the present invention is to make possible endoscope optics of the above type while making use of plastic housing parts.

In the present invention, the housing parts may be made of PPS (polyphenylenesulfide) or PPSU (polyphenyylsulfone) plastics and, nevertheless, can be hermetically joined to other housing parts made of PPS, PPSU or glass. In one connection site mode, both the plastic and the glass are coated with a metal allowing the soldering of a hermetic connection site onto it. As a result, plastic parts may be used when building the required hermetic housing, offering reductions in costs and broadening applicability. PPS and PPSU have been found practical in endoscope design, being highly temperature resistant and lending themselves to being coated well with metal for the purposes of this disclosure.

### BRIEF DESCRIPTION OF THE DRAWINGS

FIG. 1 shows the present invention in an illustrative and schematic manner in the form of an axial section of endoscope optics.

### DETAILED DESCRIPTION OF THE INVENTION

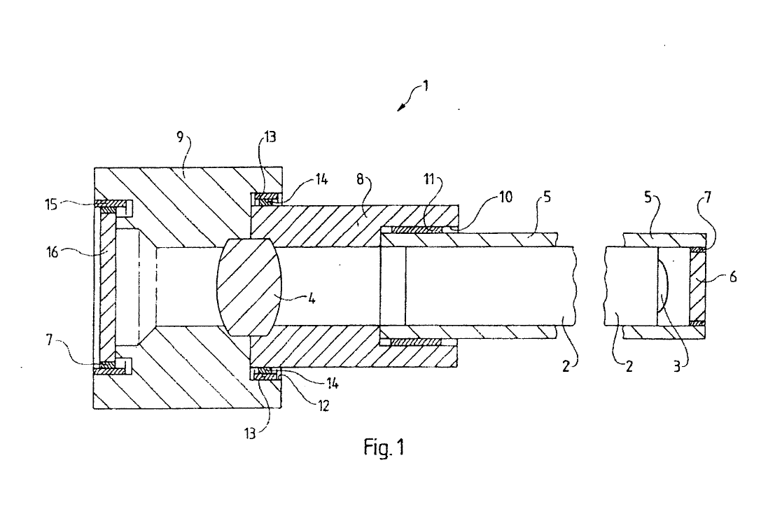

FIG. 1 shows endoscope optics 1 with an elongated image guide 2 having an objective 3 at its distal end. The image guide 2 may be a set of relay lens elements or a bundle of optic fibers, and optionally it may also be a video endoscope image guide fitted with a video camera at the objective 3 and with data wires running proximally.

In the embodiment shown, the image guide 2 is an optical image guide and is viewed on the other side of its proximal end through an ocular lens element 4.

The optical system described so far, above, is mounted in a hermetically sealed housing. This housing is fitted at its distal end zone with an elongated tube 5, which for the sake of mechanical strength is made of metal and which is sealed at its distal end by a window 6. This window is fitted at its rim with a metal coating 7, shown for illustration in a very exaggerated size in the drawing, and by means of this coating the window is hermetically soldered to the tube 5.

The tube 5 is affixed to a middle housing part 8 which in turn is affixed in a terminal housing part 9.

The two housing parts 8 and 9 are made of a plastic, namely PPS (polyphenylsulfide) or PPSU (polyphenylsulfone), that is of a plastic found effective for endoscopes. The housing part 8 is fitted at its rest surface 10 with a metal coating 11 soldered in a hermetic manner to the metal tube 5.

In the present embodiment mode, the housing part 8 is inserted into a borehole 12 of the housing part 9 at the connection site between the housing parts 8 and 9. At that connection site, the borehole 9 comprises a metal coating 13 and the outer surface of the housing part 8 comprises a metal coating 14. Again, these two metal coatings are hermetically soldered to each other. All metal coatings shown in FIG. 1 are shown exaggeratedly large.

The terminal housing part 9 is fitted at its proximal end with a metal coating 15 on the inside surface of a terminal borehole. A proximal window 16 is inserted at this site which is fitted with a metal coating 7 just like the distal window 6 is. The metal coatings 7 and 15 are hermetically soldered to each other.

As a result, the optic elements 3, 2, 4 are enclosed by a housing which is constituted by the tubular parts 5, 8, 9 and the two terminal windows 6 and 16 and which is soldered to be hermetically sealed at all connection sites.

FIG. 1 is hypothetical, strongly schematic, and didactic. For instance it omits the typical image guides used for illumination, which may be added in known manner to the design shown.

In the embodiment of FIG. 1, the plastic housing parts 8 and 9 are soldered together by means of metal coatings at all connection sites 11, 13, 15. Alternatively, however, the plastic housing parts also may be joined using technically different means. For instance, as regards the embodiment of FIG. 1, the connection at the connection sites 11 and 15 may be implemented by soldering together the metal coatings illustrated in FIG. 1. However as regards the connection site of the housing parts 8 and 9, another connection site mode, free of metal coatings and free of soldering may be selected. Illustratively, the housing parts may be joined by a direct plastic bond, for instance by fusing the plastic or by a highly sealing adhesive.

1. A rigid endoscope optics (1) comprising a housing which consists of several housing parts (5, 8, 9, 6, 16) and which hermetically encloses the optic elements (3, 2, 4) and which is fitted with hermetic connections (13, 14; 7, 15) between two housing parts (9, 8; 9, 16), wherein one (9) of the two housing parts joined to each other is made of the plastic PPS (polyphenylsulfide) or PPSU (polyphenylsulfone) and the other connected housing part (8, 16) is made of PPS or PPSU or glass, and wherein the housing parts (8, 9, 16) are fitted with a metal coating (7, 13, 14, 15) at the connection site (13, 14; 7, 15) and are soldered to each other.
